(19) United States
(12) Patent Application Publication
(10) Pub. No.: US 2025/0006303 A1
(43) Pub. Date: Jan. 2, 2025

(54) ENSEMBLE MACHINE LEARNING TECHNIQUE FOR VISUALIZING COMPLEX DATA RELATIONSHIPS

(71) Applicant: MICROSOFT TECHNOLOGY LICENSING, LLC, Redmond, WA (US)

(72) Inventors: Harsh SHRIVASTAVA, Redmond, WA (US); Robin ABRAHAM, Redmond, WA (US)

(21) Appl. No.: 18/215,801

(22) Filed: Jun. 28, 2023

(51) Int. Cl. G16B 25/10; G06N 20/20; G16B 40/20; G16B 45/00

(52) U.S. Cl. CPC G16B 25/10; G06N 20/20; G16B 40/20; G16B 45/00

(57) ABSTRACT

An ensemble machine learning model is used to visualize complex data relationships. Complex data is provided to multiple generator models that each independently create a graph representing relationships in the data. At least one of the generator models is a machine learning model that can be trained with a generator model loss function. The graphs produced by the separate generator models are combined by an ensemble model to create a consensus graph. The ensemble model may be implemented as an edge-selector neural network. The models are trained jointly with a ensemble model loss function that includes the loss functions of the generator models added as regularization terms. A visualization of the ensemble graph is created to aid a user in understanding the complex data relationships.

### BACKGROUND

Advances in machine learning and artificial intelligence are benefiting many technical fields. One use for machine learning is interpreting complex data and presenting relationships from the data visually so that it is able to be interpreted and understood by humans. Well-designed and technically accurate visual representations can be key in enabling humans, rather than computers, to understand complex data relationships.

Gene regulatory networks (GRNs) are representations of interactions between transcription factors (TFs) and their target genes. GRNs are often represented visually as graphs. In a graph visualization, nodes represent genes and edges represent interactions between the genes. The direction of an edge indicates the direction of the interaction, with an arrow pointing from the regulator gene to the target gene. The weight of an edge can be used to represent the strength of the interaction. Understanding these interactions is important for studying the mechanisms in cell differentiation, growth, and development. Although the availability of single cell ribonucleic acid (RNA)-Sequencing (scRNA-Seq) data provides unprecedented scale and resolution of gene-expression data, the inference of GRNs remains a challenge, mainly due to the complexity of the regulatory relationships and noise in the data. Computational methods are used to infer the relationships and generate putative GRNs from measured data.

Visual representations of GRNs can be used to gain insights into the structure and function of these networks. However, current techniques for generating and visualizing GRNs often do not accurately capture the underlying biological relationships. The following disclosure relates to these and other considerations.

### SUMMARY

This disclosure provides novel methods and systems for visualizing complex data relationships. The types of data discussed in this disclosure often contain many thousands of data points with subtle indications of relationships and potentially contradictory signals. Absent appropriate processing and presentation, the relationships captured by the data may remain obscure or unappreciated.

This disclosure provides an ensemble approach that uses machine learning for recovery, analysis, and visualization of GRNs. Artificial intelligence and machine learning techniques are used to generate graphs that visually represent GRNs. The GRNs are generated from gene expression data. Gene expression data can be obtained from microarray analysis of RNA expression data. However, RNA expression data is difficult or impossible to interpret without extensive analysis and processing.

The gene expression data is initially processed by passing it through multiple separate GRN recovery methods. These recovery methods can be referred to as “generator models” that may be machine learning models or other types of non-learnable computer models. Each generator model attempts to discover relationships in the gene expression data. The relationships are typically those of transcription factors regulating the expression of other genes. These relationships are represented visually as graphs having nodes and edges. Thus, each generator model will create a different graph from the same gene expression data.

These multiple graphs can show alternative and conflicting ways of interpreting the same gene expression data. The multiple graphs are then provided to an ensemble model which is a machine learning model. The machine learning model combines multiple graphs to create consensus relationship data that represents a consensus view of the relationships captured in the graphs from the generator models. The consensus relationship data is visualized to generate a single consensus graph. The consensus graph represents the relationships between genes captured in the gene expression data better than any of the individual graphs created by the generator models. The ensemble model may be implemented as an edge-selector neural network which compares the edges of the input graphs from the generator models and learns a potentially complex function to determine if there should be an edge (representing a regulatory relationship) between any two nodes.

Although the generator models and the ensemble model are separate models, the techniques of this disclosure create a single end-to-end model that is jointly trained. Some of the generator models are machine learning models which are trainable with their respective loss functions. The ensemble model is also trainable with its own loss function. Instead of training each type of model separately, they are trained together with the loss functions of the generator models used as regularization terms in the loss function of the ensemble model. Using the loss functions of the generator models as regularization terms allows the generator models to retain their functionality as separate stand-alone models.

In order to generate sufficient data for training and capturing the underlying distribution of the family of GRNs, biological data simulators that model the kinetic equations that govern the relations between transcription factors and other genes are used to generate simulated data in form of (GRN, expression data) pairs. The simulated data may be supplemented with experimentally derived data that captures known regulatory relationships between genes. Additionally, any known ground-truth relationships (e.g., from experimental data) can be included directly in the simulator settings so that the simulated data includes those known relationships. The simulated data and any experimentally derived data added to the training data set provides a ground truth that is used for training the models to minimize the loss function. Because the two types of models—generator and ensemble—are entangled due to the way they are trained, both become optimized through this training technique. Each separate generator model may have different strengths such as greater accuracy for certain types of tissues or better resilience to certain types of noise. Combining multiple generator models, and training them jointly with an ensemble model, creates a machine learning model that is more generalizable and more accurate than any of the models individually. The resulting GRNs generated by this new machine learning model can then be visualized as graphs and used to better understand the structure and function of gene regulatory relationships.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 is a diagram that illustrates the use of a machine learning model to generate a visual representation of complex data.

FIG. 2 is a diagram that illustrates a pipeline starting with a microarray and ending with a graph that visually represents a GRN.

FIG. 3 is a computer architecture diagram of an illustrative computing device capable of implementing aspects of the techniques and technologies of this disclosure.

FIG. 4 is a flow diagram of an illustrative method for using a machine learning model to generate a consensus graph from gene expression data.

FIG. 5 is a flow diagram of an illustrative method for jointly training generator models and an ensemble model.

### DETAILED DESCRIPTION

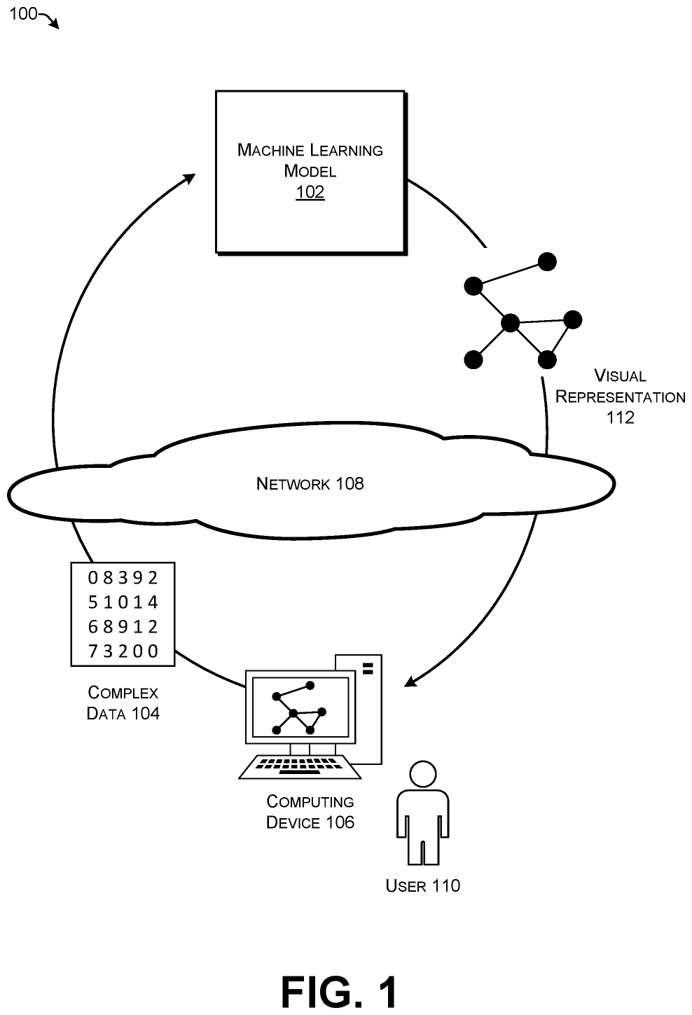

FIG. 1 is a diagram 100 that illustrates at a high-level the use of a machine learning model 102 to visualize complex data relationships. The machine learning model 102 may be implemented as any type of machine learning model such as a neural network including deep learning techniques. The specific type and architecture of the machine learning model 102 may be varied based on the type of complex data 104 and the underlying relationships captured by that complex data 104. One type of complex data 104 that may be processed by the machine learning model 102 is gene expression data.

The complex data 104 may be provided by a computing device 106 that is physically separate from other computer systems which host machine learning model 102. Thus, in one of implementation, the machine learning model 102 may be implemented in the “cloud” and accessed by the computing device 106 via a network 108 such as, but not limited to, the Internet. However, in other implementations, the machine learning model 102 may be hosted on the same computing device 106 that provides the complex data 104.

The complex data 104 may be thought of as “raw data” generated by other devices or collected through experiment procedures. While the complex data 104 can be interpreted to identify relationships captured by the data, it generally is not interpretable by a human user 110. Thus, complex data 104 represents data that in its current form is not usable or meaningful to a user 110.

The machine learning model 102 can both identify relationships in the complex data 104 and generate a visual representation 112 of those relationships. The visualization of 112 may be, but is not limited to, a graph. The machine learning model 102 is trained off-line using training data to learn how to generate an accurate visual representation 112 from complex data 104. The visual representation 112 generated by the machine learning model 102 is provided to the computing device 106. The visual representation 112 may be communicated via the network 108 if the computer systems hosting the machine learning model 102 are located remote from the computing device 106. The visual representation 112 may be rendered on a display device of the computing device 106 so that the user 110 is able to perceive and understand relationships present in the complex data 104. The techniques of this disclosure improve usability of the complex data 104 by the user 110 through the generation of a technically accurate visual representation 112.

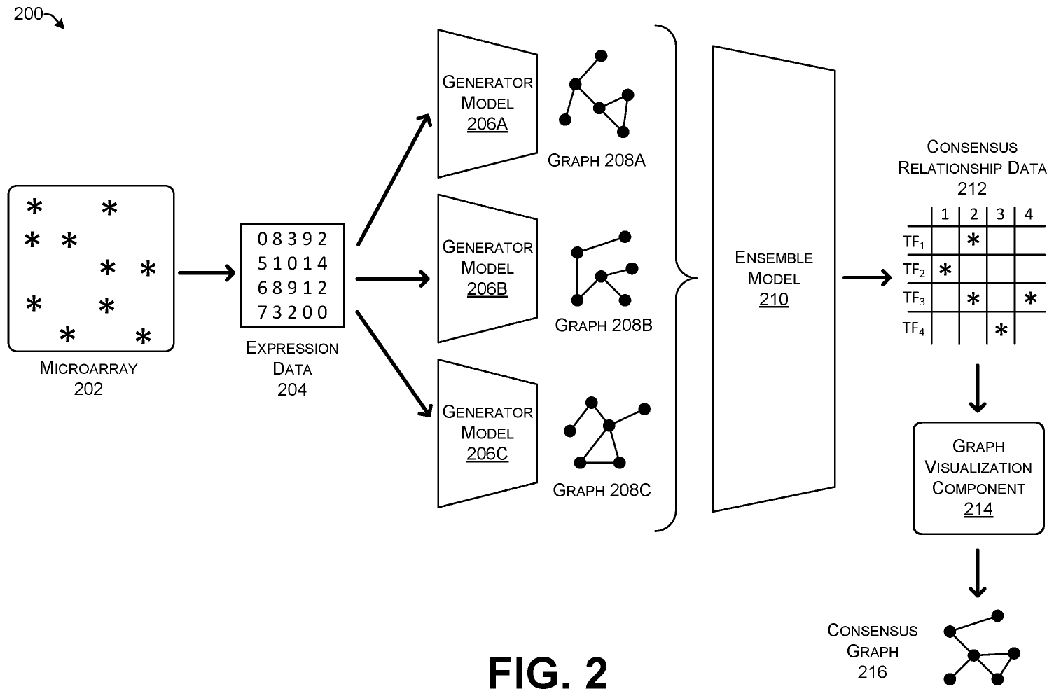

FIG. 2 is a diagram 200 that illustrates a pipeline which uses machine learning models to generate a visual representation of complex data relationships. The pipeline may begin with a microarray 202. A microarray 202 is a laboratory tool used to detect the expression of thousands of genes at the same time. It is typically a small glass slide that is coated with thousands of tiny spots, each of which contains a known deoxyribonucleic acid (DNA) sequence or gene. The DNA molecules attached to each spot act as probes to detect gene expression.

The microarray 202 is used to generate gene expression data 204. The gene expression data 204 may come from scRNA-Seq or bulk sequencing. A microarray 202 is used to generate gene expression data 204 by extracting RNA from a sample of cells or tissue. The RNA is then converted into cDNA, which is a single-stranded DNA molecule that is complementary to the RNA. The cDNA is then labeled with a fluorescent dye. The labeled cDNA is then hybridized to DNA spots attached to the microarray 202. Hybridization is the process in which two complementary DNA sequences bind to each other. In this case, the cDNA molecules will bind to the DNA probes on the microarray. The more cDNA molecules that bind to a particular probe, the more active that gene is in the sample. After hybridization, the microarray is scanned to measure the fluorescence of each spot. The intensity of the fluorescence indicates the level of gene expression for that gene. The fluorescence signals are processed to normalize the signal values across the arrays. Any standard scaler or conventional technique for normalization may be used.

The gene expression data 204 may contain data from multiple different microarrays 202. In some implementations, the gene expression data 204 may be in the form of a matrix with rows representing genes (e.g., G₁, G₂, G₃, . . . , G_{N}) and columns representing samples (S₁, S₂, S₃, . . . , S_{N}) such as samples of different tissue types or from different organisms. Thus, the gene expression data 204 may contain S_{N} samples and G_{N} features. One example of expression data 204 is a CEL file. A CEL file is a data file created by Affymetrix DNA microarray image analysis software. It contains the data extracted from “probes” on an Affymetrix GeneChip and can store thousands of data points.

The gene expression data 204 is provided to multiple generator models 206. Although three generator models 206A, 206B, and 260 are illustrated, there may be any number of two or more generator models 206. The generator models 206 are GRN recovery models and may also be referred to as graph recovery models. Each generator model 206 takes gene expression data 204 as an input and outputs a graph that represents a GRN.

There are many different techniques for GRN recovery. Generator models 206 may use regression-based techniques, partial correlation, graphical lasso, Markov networks, directed acyclic graphs (DAGs), Bayesian networks, stochastic embedding (SEM), or other techniques for graph recovery. The methods may be supervised or unsupervised. In some implementations, unsupervised methods are preferred. Some of the genes observed on the microarray 202 are transcription factors. Transcription factors are genes that code for proteins which regulate the expression of other genes. Thus, transcription factors have a key role in gene regulatory networks. Generally, it is known which genes are transcription factors. Creating a gene regular network involves identifying the other genes regulated by each transcription factor. This is done by trying to find sparse connections between each transcription factor and a subset of the other genes. One object of GRN recovery is to find which transcription factors are most important for regulation of a given gene. This can be done by fitting a regression for every gene based on the transcription factors.

At least one of the generator models 206 is trainable meaning that it is a machine learning model that can be trained using a loss function referred to as a generator loss function. A trainable generator model 206 is a deep model that is end-to-end differentiable. Other generator models 206 may or may not be trainable. A generator model 206 that is not trainable is a fixed model such as a linear model or other type of GRN recovery model that cannot be trained using a loss function. With a non-trainable generator model 206 there is no way to propagate loss and no weights that can be updated based on training. For example, a first generator model 206A may be a trainable model and a second generator model 206B may be a model that is not trainable.

Examples of trainable generator models 206 include GLAD, uGLAD, Neural Graph Revealers (NGR), and GRNUlar. For descriptions of these models see Harsh Shrivastava et al. GLAD: Learning sparse graph recovery. In International Conference on Learning Representations (2020); Harsh Shrivastava et al. uGLAD: Sparse graph recovery by optimizing deep unrolled networks. arXiv: 2205.11610v2 (2022); Harsh Shrivastava and Urszula Chajewska. Neural Graph Revealers. arXiv: 2302.13582v2 (2023 Harsh Shrivastava et al. GRNUlar: A Deep Learning Framework for Recovering Single-Cell Gene Regulatory Networks. J. of Computational Biology. 29 (1) 27-44 (2022). Examples of generator models 206 that are not trainable include a simple regression-based model, GENIE3, and GRNBoost2. For descriptions of these models see Huynh-Thu VA et al., Inferring Regulatory Networks from Expression Data Using Tree-Based Methods. PLoS ONE 5(9):e12776 (2010). Any or all of the models mentioned above may be used. However, machine learning system of this disclosure is not limited to use of any particular type of generator model 206 and may use models other than those listed here.

Each of the generator models 206A-C creates a corresponding graph 208A-C. Generally, different models 206 will create somewhat different graphs 208 from the same expression data 204. However, it is possible that two different generator models 206, particularly two models that are similar, could create the same graph 208. In each of the graphs 208, the nodes represent genes including transcription factors. Regulatory relationships between the genes are shown by the edges in the graphs 208. Each of the graphs 208A-C may be a directed graph or an undirected graph. In a directed graph, the edges show the direction of regulation generally going from a transcription factor to genes that are regulated by the transcription factor. Transcription factors can regulate other transcription factors thus there may be an edge between nodes representing two transcription factors. For some generator models 206, the resulting graph 208 may also include weights for the edges indicating the strength of the relationship between the genes.

The graphs 208A-C represent an intermediate output of the system. Each of the graphs 208A-C is a valid GRN. The graphs 208A-C created by the generator models 206A-C remain available for presentation to a user even though they are further processed by the ensemble model 210 as described below.

The ensemble model 210 finds a consensus among the graphs 208A-C created by the generator models 206A-C. Ensemble models are a machine learning approach to combine multiple other models. Ensemble models offer a solution to overcome the technical challenges of building a single model. In this application, discovering and visualizing GRN's, the ensemble model 210 can make use of multiple known generator models 206A-C. The ensemble model 210 may be implemented as an edge-selector neural network that determines for any two nodes (i.e., genes in the network) if they are connected by an edge. If the graphs 208A-C provided as input to the ensemble model 210 are directional and/or have weighted edges then the ensemble model 210 may also generate a consensus from the directions and the weights. The ensemble model 210 is a machine learning model that learns a function over the predictions made by the generator models 206A-C. One example of a suitable ensemble model 210 is EnGRaiN which is described in Maneesha Alum et al. EnGRaiN: a supervised ensemble learning method for recovery of large-scale gene regulatory networks, Bioinformatics, 38 (5) 1312-1319 (2022).

The ensemble model 210 generates consensus relationship data 212 that represents relationships between transcription factors and other genes in the expression data 204. The consensus relationship data 212 is identified through processing of the graphs 208A-C created by the generator models 206A-C. The consensus relationship data 212 may include directional relationships as well as strengths of the connections if that information was provided by the generator models 206A-C.

The consensus relationship data 212 is provided to a graph visualization component 214 that generates a consensus graph 216. The consensus graph 216 is a visual representation of the GRN inferred from the expression data 204. The graph visualization component 214 determines how nodes representing genes and edges representing relationships between those genes are arranged in a graphical presentation. For example, the graph visualization component 214 may use a different color or visual characteristic to distinguish transcription factors from other genes. The graph visualization component 214 may also arrange the nodes in a way that minimizes the number of edges which cross over other edges.

The consensus graph 216 is similar to the graphs 208A-C created by the generator models 206A-C because it represents the genes as nodes and regulatory relationships between those genes as edges. However, the specific structure of the consensus graph 216 is likely different than any of the graphs 208A-C. Thus, the consensus graph 216 represents a new visual representation of the expression data 204 that did not previously exist. Because the ensemble model 210 combines outputs from multiple different generator models 206 to create a single GRN, the consensus graph 216 more accurately represents the biological relationships between the genes than any of the graphs 208A-C and produces a more generalizable model.

The machine learning model 102 shown in FIG. 1 may include the generator models 206A-C, ensemble model 210, and the graph visualization component 214. All of these parts may function together as a single machine learning model that takes gene expression data 204 is an input and provides a consensus graph 216 representing a high-fidelity GRN as output.

### Illustrative Computing Architecture

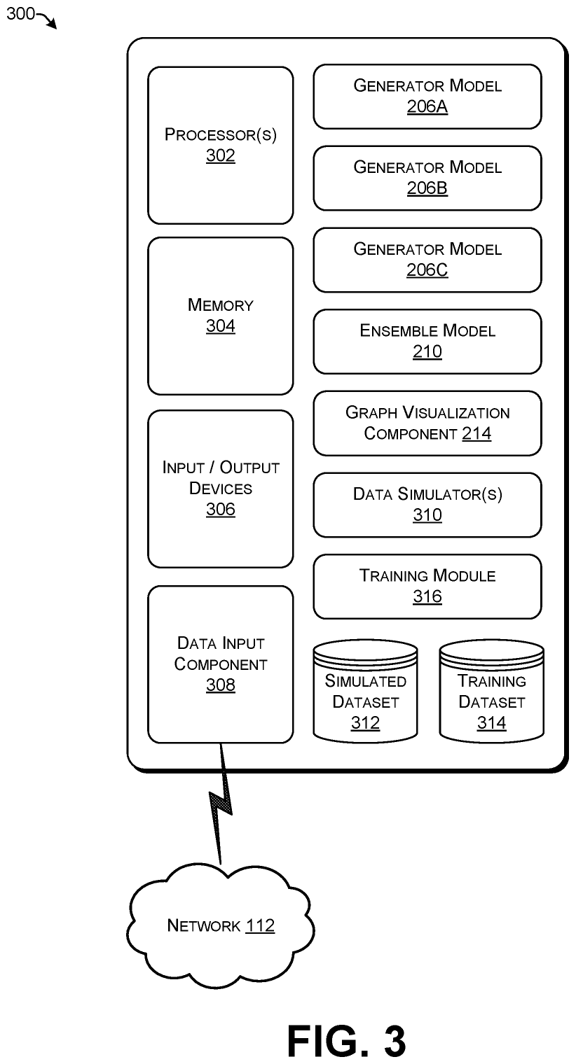

FIG. 3 shows a block diagram of an illustrative computing device 300 that may be used to implement the machine learning model 102 introduced in FIG. 1 as well as the generator models 206A-C and ensemble model 210 shown FIG. 2. None, some, or all of the components of the computing device 300 shown in FIG. 3 may be implemented in the computing device 106 shown in FIG. 1.

The computing device 300 may include one or more processor(s) 302 and memory 304 also referred to as computer-readable media, both of which may be distributed across one or more physical or logical locations. The processor(s) 302 may include any combination of central processing units (CPUs), graphical processing units (GPUs), single core processors, multi-core processors, processor clusters, application-specific integrated circuits (ASICs), programmable circuits such as Field Programmable Gate Arrays (FPGA), and the like. In one implementation, one or more of the processor(s) 302 may use Single Instruction Multiple Data (SIMD) or Single Program Multiple Data (SPMD) parallel architectures. For example, the processors(s) 302 may include one or more GPUs or CPUs that implement SIMD or SPMD. A first set of processor(s) 302 may be used for training the machine learning model 102 such as, for example, tens or hundreds of GPUs. And a second set of one or more processors(s) 302, such as one or more CPUs, may be used for passing inputs through the machine learning model 102 once trained.

One or more of the processor(s) 302 may be implemented in software and/or firmware in addition to hardware implementations. Software or firmware implementations of the processor(s) 302 may include computer- or machine-executable instructions written in any suitable programming language to perform the various functions described. Software implementations of the processor(s) 302 may be stored in whole or part in the memory 304.

Alternatively or additionally, the functionality of computing device 300 can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The memory 304 of the computing device 300 may include removable storage, non-removable storage, local storage, and/or remote storage to provide storage of computer-readable instructions, data structures, program modules, and other data. The memory 304 is coupled to the processor(s) 302. Computer-readable media includes at least two types of media: computer-readable storage media and communications media. Computer-readable storage media includes volatile and non-volatile, removable and non-removable media implemented in any method or technology for storage of information such as computer-readable instructions, data structures, program modules, or other data. Computer-readable storage media includes, but is not limited to, RAM, ROM, EEPROM, flash memory, solid-state storage or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage media does not include communication media. Thus, computer-readable storage media excludes media consisting solely of a modulated data signal, a carrier wave, or a propagated signal, per se.

The computing device 300 may include one or more input/output devices 306 such as a keyboard, a pointing device, a touchscreen, a microphone, a camera, a display, a speaker, a printer, and the like. Input/output devices 306 that are physically remote from the processor(s) 302 and the memory 304 (e.g., the monitor and keyboard of a thin client) are also included within the scope of the input/output devices 306.

A data input component 308 may also be included in the computing device 300. The data input component 308 can be implemented as a network interface or other point of interconnection between the computing device 300 and a network 108. The data input component 308 may be implemented in hardware for example as a network interface card (NIC), a network adapter, a LAN adapter or physical network interface. The data input component 308 can be implemented in part in software. The data input component 308 may be implemented as an expansion card or as part of a motherboard. The data input component 308 may implement electronic circuitry to communicate using a specific physical layer and data link layer standard such as Ethernet, InfiniBand, or Wi-Fi. The data input component 308 may support wired and/or wireless communication. The data input component 308 provides a base for a full network protocol stack, allowing communication among groups of computers on the same local area network (LAN) and large-scale network communications through routable protocols, such as Internet Protocol (IP). The data input component 308 may be configured to receive gene expression data such as microarray data.

The network 108 may be implemented as any type of communications network such as a local area network, a wide area network, a mesh network, an ad hoc network, a peer-to-peer network, the Internet, a cable network, a telephone network, and the like.

The computing device 300 includes multiple modules that may be implemented as instructions stored in the memory 304 and executed by the processor(s) 302 and/or implemented, in whole or in part, by one or more hardware logic components or firmware. These modules may include components of the machine learning model 102 introduced in FIG. 1 or components of the pipeline shown in FIG. 2 such as a plurality of generator models 206A-C, the ensemble model 210, and the graph visualization component 214. Each of the generator models 206A-C is configured to generate a graph representing relationships in the gene expression data. The generator models 206A-C may be graph recovery models. The ensemble model 210 is configured to create consensus relationship data from the graphs generated by the plurality of generator models 206A-C. The ensemble model 210 can learn a function for each edge in the consensus graph over the edges present in the graphs generated by the plurality of generator models 206A-C.

The computing device 300 may also include one or more biological data simulator(s) 310 that are configured to generate a simulated dataset 312 that includes a simulated graph and a simulated gene expression data. However, in other implementations the data simulator(s) 310 may be located elsewhere and simulated data provided to the computing device 300 via the data input component 308. There are many examples of biological data simulators 310 known to those of skill in the art. The biological data simulator(s) 310 capture biological insights in the form of kinetic equations. The biological data simulators 310 are used to generate a simulated graph and simulated gene expression data. Simulation can be done for a variety of tissues and cell types to create multiple sets of simulated graphs with the accompanying simulated gene expression data. For example, a first biological data simulator 310 can simulate data from mouse embryonic stem cells (MESCs) while a second biological data simulator 310 simulates data from human embryonic stem cells (hESCs).

The biological data simulator(s) 310 can simulate a variety of biological relationships, ambient conditions, and technical noise that can occur while performing experiments. The biological data simulator(s) 310 may also add simulated noise of the type that would be found in experimentally generated gene expression data. Thus, the biological data simulator(s) 310 can generate biologically plausible samples having a similar statistical distribution to experimentally generated data. The simulated data generated by one or more biological data simulators 310 is collected in the simulated dataset 312. The simulated dataset 312 is useful for training machine learning models because there is a known correlation between the simulated gene expression data and the simulated graph. This provides a ground truth for training a loss function. Due to the sparseness of experimentally derived information identifying gene regulatory relationships, there are many genes and interactions between genes for which the biological ground truth is not known. Thus, the simulated dataset 312 can be used for training.

Some examples of current biological data simulators include, but are not limited to, BEELINE, SYNTREN, and SERGIO. For additional description of these simulators see Aditya Pratapa et al. Benchmarking algorithms for gene regulatory network inference from single-cell transcriptomic data. Nat Methods 17, 147-154 (2020); Van den Bulcke, T. et al. SynTReN: a generator of synthetic gene expression data for design and analysis of structure learning algorithms. BMC Bioiformatics 7, 43 (2006); Payam Dibaeinia and Saurabh Sinha. SERGIO: A Single-Cell Expression Simulator Guided by Gene Regulatory Networks. Cell Systems 11 (3), 257-271 (2020). However, biological data simulators other than those listed here including later developed biological data simulators may be used.

Data from the simulated dataset 312 may be used alone or in combination with other data to create a training dataset 314. The other data may be experimentally identified relationships between genes. This may come from mining published scientific papers or from performing benchtop experimentation. Known regulatory relationships, and lack of relationships, between transcription factors and genes may also be added on a gene-per-gene basis. Experimentally identified regulatory relationships between genes are a biological ground truth that can be useful to include in the training dataset 314. This data representing known biological ground truth may be given greater weight during training. Thus, the training dataset 314 includes the simulated dataset 312 and may, but does not necessarily, include additional training data.

The computing device 300 also includes a training module 316 configured to jointly training any trainable generator models among the generator models 206A-C and the ensemble model 210. The training module 316 uses the loss function from the ensemble model 210 and the generator loss functions from the generator models 206A-C that are trainable models which themselves have loss functions. If there is a generator model 206 that is not trainable, such as a static model, it is not trained by the training module 316. There may also be generator models 206 that are machine learning models with loss functions that cannot be merged with the loss function of the ensemble model 210. These types of generator models 206, even though they are machine learning models, are not jointly trained by the training module 316.

The generator loss functions from those generator models 206 that are trainable are combined with the loss function of the ensemble model 210 by adding them as regularization terms. Regularization refers to modifying the loss function to penalize certain values of the weights that the model is learning. This creates a single loss function that can be used for end-to-end training. This loss function is the loss function of the ensemble model 210 plus multiple regularization terms from each of the trainable generator models 206.

Combining the loss function of the ensemble model 210 and the generator loss functions by adding them as regularization terms keeps the overall loss function optimization stable. Each of the generator models 206 retains its own intrinsic design and is still able to generate a valid GRN after joint training. Thus, intermediate graphs produced by the separate generator models 206 may be presented to the user together with the final consensus graph. Showing the consensus graph together with intermediate graphs allows a user to better understand how the consensus graph was generated. This improves transparency and interpretability of the consensus graph.

Joint training allows the ensemble model 210 and plurality of generator models 206 to function as a single machine learning model. Yet it is still possible to use any of the generator models 206 independently. This combination of models and techniques for training creates an combined machine learning model that is much more generalizable than any of the individual generator models 206A-C. The machine learning model that combines these different models is able to generate graphs from many types of gene expression data such as data from a wide range of microarrays and from different tissue types. The overall machine learning model can also be improved by off-line training with a large amount of simulated data.

The training may be performed by any conventional machine learning training technique. The training dataset 314 is used for the joint training performed by the training module 316. The weights of the models are learned in a way that minimizes the difference between the predicted graph and the ground truth graph—minimizing the loss. Specifically, training can be used to establish the weights for the neural network used by the ensemble model 210. The machine learning model may be trained in a way that minimizes both loss and generalization loss to avoid overfitting. In one implementation, training is performed using stochastic gradient descent. For example, training may be performed using the Adam optimizer which is a stochastic gradient descent method that is based on adaptive estimation of first-order and second-order moments. For a description of the Adam optimizer see Kingma, Diederik P. and Jimmy Ba. “Adam: A Method for Stochastic Optimization.” CoRR abs/1412.6980 (2014): n. pag.

### Illustrative Methods

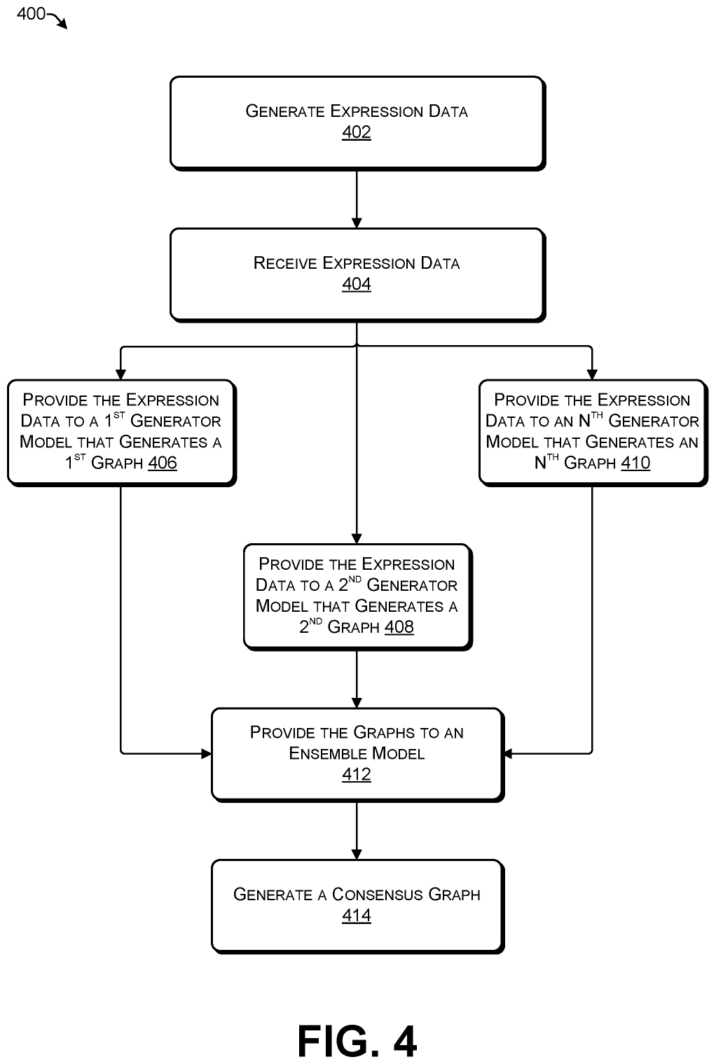

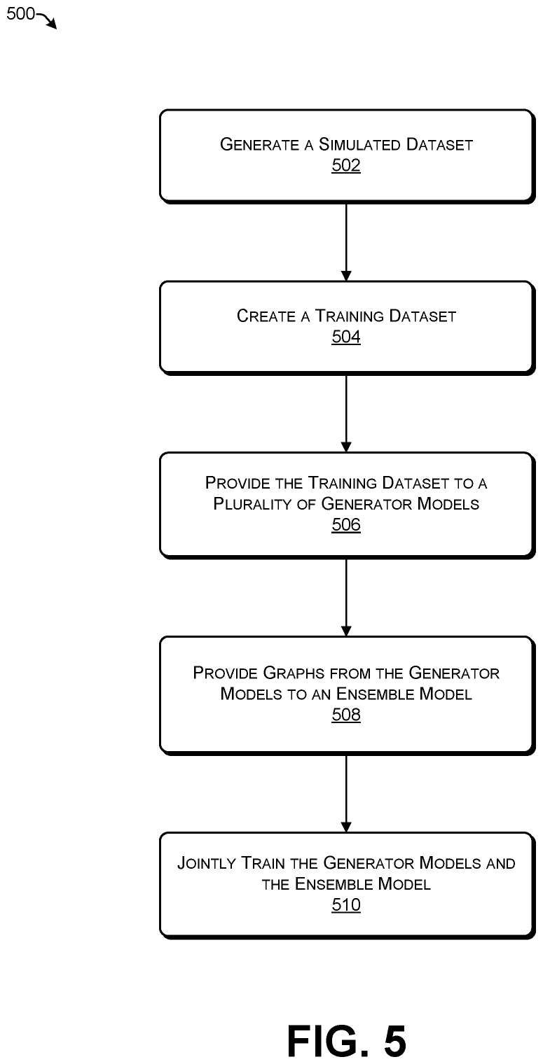

For ease of understanding, the processes discussed in FIGS. 4 and 5 are delineated as separate operations represented as independent blocks. However, these separately delineated operations should not be construed as necessarily order dependent in their performance. The order in which a process is described is not intended to be construed as a limitation, and any number of the described process blocks may be combined in any order to implement the process or an alternate process. Moreover, it is also possible that one or more of the provided operations is modified or omitted.

FIG. 4 is a flow diagram of an illustrative method 400 for using a machine learning model to generate a consensus graph from gene expression data. Method 400 may be implemented with the pipeline shown in FIG. 2 and the computing device shown in FIG. 3.

At operation 402, gene expression data is generated. The gene expression data may be generated by any known technique for capturing gene expression activity. For example, the gene expression data may be generated with a microarray. Techniques for generating gene expression data with a microarray are well known to those of ordinary skill in the art. The gene expression data may be normalized using any suitable technique for normalizing data such as microarray expression data. The gene expression data may be microarray data that is generated by single cell RNA sequencing or bulk sequencing.

At operation 404, the gene expression data generated at operation 402 is received. The gene expression data may be received by a data input component of a computing device such as the computing device shown in FIG. 3. The gene expression data may be received as a computer file for example as a CEL file that is used to convey gene expression data.

At operation 406, the gene expression data is provided to a first generator model that generates a first graph. The first graph represents relationships in the gene expression data such as regulatory relationships between transcription factors and genes. The first generator model may be an example of trainable generator model that is end-to-end differentiable and that was previously trained with a generator model loss function. Thus, the first generator model may be a machine learning model such as a neural network. There are many possible models that may be used, each with its own loss function. Examples of trainable generator models include, but are not limited to, GLAD, uGLAD, Neural Graph Revealers (NGR), and GRNUlar.

At operation 408, the gene expression data is provided to a second generator model that generates a second graph. The second generator models may be a different type of model than the first generator model. For example, the second generator model may be a fixed model that is not trainable. Thus, the second generator model may be a type of mathematical model that does not use machine learning and is not trained with a loss function. Examples of fixed generator models include, but are not limited to, GENIE3 and GRNBoost2. Although the second generator model is described as a fixed model in method 400, the methods of this disclosure can be used without a fixed generator model. They may also be used with more than one fixed generator model.

At operation 410, the gene expression data is provided to a third generator model that generates a third graph. The third generator model may be any of the types of generator models described above for the first generator model or the second generator model. Although three generator models are described in method 400, the methods of this disclosure may use as few as two generator models or more than three generator models. There is no upper limit on the possible number of generator models that can be used.

At operation 412, the graphs generated by the generator models at operations 406, 408, and 410 are provided to an ensemble model. The ensemble model is a machine learning model that creates consensus relationship data from the graphs generated by the generator models. Thus, in this example method, it takes as input the first graph, the second graph, and the third graph. The ensemble model learns a function for each edge in the consensus graph over the edges present in the input graphs (i.e., the first graph, the second graph, and the third graph). Thus, the ensemble model may be implemented as an edge-selector neural network. One example of a suitable ensemble model is EnGRaiN.

The ensemble model and the first generator model (as well as the third generator model if it is a trainable model) are jointly trained with an ensemble model loss function (which is the loss function of the ensemble model) that includes the generator model loss function of the first generator model as a regularization term. If the third generator model is also a trainable model, the generator model loss function of that model is also added as a second regularization term to the ensemble model loss function. This joint end-to-end training of multiple models together creates a single machine learning model. The weights of connections in the neural network of the ensemble model is learned using the regularization terms. Thus, the selection of generator models and the behavior of those models affects the creation of the neural network used for the ensemble model. Additionally, the joint training allows signals that can be used to improve the overall machine learning model to be propagated to the generator models.

At operation 414, a consensus graph is generated. The consensus graph is a visualization of the consensus relationship data generated by the ensemble model at operation 412. The consensus relationship data is processed to identify nodes and edges which are shown in the consensus graph. The consensus graph represents the relationships of a GRN in a way that is more accurate than any of the graphs created by the first generator model, the second generator model, and the third generator model. In the consensus graph (as well as the graphs created by the first generator model, the second generator model, and the third generator model) the nodes represent genes and the edges represents a regulatory relationship between a first one of the genes and a second one of the genes. At least one of the genes is a transcription factor. The second one of the genes may be a second transcription factor or it may be a different type of gene.

FIG. 5 is a flow diagram of an illustrative method 500 for training a machine learning model to generate a visual representation of complex data. Method 500 may be implemented with the pipeline shown in FIG. 2 and the computing device shown in FIG. 3.

At operation 502, a simulated dataset is generated. The simulated dataset may be generated with a biological data simulator as described above in FIG. 3. The simulated dataset includes simulated gene expression data and a simulated graph representing relationships in the simulated gene expression data. The relationships may be regulatory relationships between transcription factors and genes. The biological data simulator can generate the simulated data set using kinetic equations. For example, the biological data simulator may be, but is not limited to, any one of BEELINE, SYNTREN, or SERGIO.

The simulated dataset may contain simulated data from multiple different biological data simulators. The simulated data set may contain simulated data from a biological data simulator generating simulated data that simulates a variety of different conditions and cell types. Thus, the simulated data set may contain a large amount of simulated data generated both from multiple different biological data simulators as well as from an individual biological data simulator generating data in response to a range of different settings. Using a training dataset with data from a variety of different sources makes the models trained from this training dataset more generalizable to a wide variety of different types of real-world data.

At operation 504, a training dataset is created. The training dataset includes at least a portion may include all of the simulated data set. The training dataset may also include training data from sources other than the simulated dataset. For example, the training dataset may include data from at least one experimental identified relationship such as a regulatory relationship between a transcription factor and a gene. Experimental identified relationships may be obtained from mining existing scientific literature or performing experiments to observe the interactions of transcription factors and genes. Data that comes from an experiment may be assumed to be more accurate than simulated data and given greater weight in the training data set.

At operation 506, the training dataset is provided to a plurality of generator models. Each of the generator models creates a separate graph representing relationships in the training data. Thus, there are a plurality of graphs created. At least one of the plurality of generator models is a trainable generator model that is end-to-end differentiable and was previously trained with a generator model loss function. The plurality of generator models may include any number of other generator models such as additional trainable generator models and/or linear models that are not trainable. The generator models may be, but are not limited to, any of the types of generator models described above such as GLAD, uGLAD, Neural Graph Revealers (NGR), GRNUlar, GENIE3, or GRNBoost2.

At operation 508, the graphs created by the plurality of generator models are provided to an ensemble model. The ensemble model is a machine learning model that creates a consensus graph from the graphs created by the plurality of generator models. The ensemble model may create the consensus graph by first creating consensus relationship data and then generating the consensus graph to visualize the consensus relationship data. The ensemble model may be a machine learning model such as a neural network that is trained with a loss function referred to as an ensemble model loss function. One example of a suitable ensemble model is EnGRaiN.

At operation 510, the generator models and ensemble model are jointly trained on the training dataset. Training may be performed using the ensemble loss function that includes the loss functions from any trainable generator models as regularization terms. The loss function for the ensemble model may be set as loss value 0 if an identified edge is correct and a loss of 1 for each edge that is not recovered correctly. The training works to minimize the difference between consensus graphs generated by the ensemble model and simulated graphs in the training dataset. The training may be performed by any conventional machine learning training technique such as stochastic gradient descent. Using the loss functions from trainable generator models as regularization terms allows the generated models to be updated during training while also retaining their ability to function independently and generate valid GRNs separate from the ensemble model. This provides end-to-end learning for the generator models and the ensemble model so that they can function as a single machine learning model.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A method for visualizing complex data relationships comprising: receiving gene expression data (204); providing the gene expression data (204) to a first generator model (206A) that generates a first graph (208A) representing relationships in the gene expression data (204), wherein the first generator model (206A) is a trainable generator model that is end-to-end differentiable and was previously trained with a generator model loss function; providing the gene expression data (204) to a second generator model (206B) that, in parallel with the first generator model (206A), generates a second graph (208B) representing relationships in the gene expression data (204);providing the first graph (208A) and the second graph (208B) to an ensemble model (210) that is a machine learning model (102) and creates consensus relationship data (212) from the first graph (208A) and the second graph (208B), wherein the ensemble model (210) and the first generator model (206A) are jointly trained with an ensemble model loss function that includes the generator model loss function as a regularization term; and generating a consensus graph (216) having nodes and edges from the consensus relationship data (212).

Clause 2. The method of clause 1, wherein the gene expression data (204) is microarray data generated by single cell RNA sequencing or bulk sequencing.

Clause 3. The method of clause 1 or 2, further comprising generating gene expression data (204) with a microarray (202).

Clause 4. The method of any of clauses 1-3, wherein the first generator model (206A) is one of GLAD, uGLAD, Neural Graph Revealers (NGR), or GRNUlar.

Clause 5. The method of any of clauses 1-4, wherein the second generator model (206B) is a fixed model that is not trainable.

Clause 6. The method of clause 5, wherein the second generator model is one of GENIE3 or GRNBoost2.

Clause 7. The method of any of clauses 1-6, wherein the ensemble model (210) learns a function for each edge in the consensus graph (216) over the edges present in the first graph (208A) and the second graph (208B).

Clause 8. The method of any of clauses 1-7, wherein the ensemble model is an edge-selector neural network.

Clause 9. The method of any of clauses 1-8, wherein, in the consensus graph (216), the nodes represent genes and the edges represents a regulatory relationship between a first one of the genes and a second one of the genes, wherein the first one of the genes is a transcription factor gene.

Clause 10. Computer-readable storage media comprising instructions that when executed by a processor cause a computing device to perform the method of any of clauses 1-8.

Clause 11. A system for visualizing complex data relationships comprising: a processor (302); memory (304) coupled to the processor (302); a data input component (308) configured to receive gene expression data (204); a plurality of generator models (206) each configured to generate a graph (208) representing relationships in the gene expression data (204), wherein at least one of the plurality of generator models (206) is a trainable generator model (206A) that is end-to-end differentiable and was previously trained with a generator model loss function; an ensemble model (210) that is configured to create consensus relationship data (212) from the graphs (208) generated by the plurality of generator models (206), wherein the ensemble model (210) is a machine learning model (102) trained with an ensemble model loss function that includes the generator model loss function as a regularization term; and a graph visualization component (214) configured to generate a consensus graph (216) having nodes and edges from the consensus relationship data (212).

Clause 12. The system of clause 11, further comprising a biological data simulator (310) configured to generate a simulated dataset (312) that includes a simulated graph and simulated gene expression data for use as a training dataset (314).

Clause 13. The system of clause 11 or 12, further comprising a training module (316) configured to jointly train the trainable generator model (206A) and the ensemble model (210) on the training dataset (314) by minimizing the loss of the ensemble model loss function that includes the generator model loss function as a regularization term.

Clause 14. The system of any of clauses 11-13, wherein the plurality of generator models (206) includes a first trainable generator model (206A) that was previously trained with a first generator model loss function and a second trainable generator model (206C) that was previously trained with a second generator model loss function and the ensemble model (210) is trained with an ensemble model loss function that includes the first generator model loss function as a first regularization term and the second generator model loss function as a second regularization term.

Clause 15. The system of any of clauses 11-14, wherein the plurality of generator models (206) are graph recovery models.

Clause 16. The system of any of clauses 11-15, wherein the ensemble model (210) learns a function for each edge in the consensus graph (216) over the edges present in the graphs (208) generated by the plurality of generator models (206).

Clause 17. A method for training a machine learning model (102) to generate a visual representation (112) of complex data (104) comprising: generating a simulated dataset (312) with a biological data simulator (310), wherein the simulated dataset (312) includes simulated gene expression data and a simulated graph representing relationships in the simulated gene expression data; creating a training dataset (314) that comprises at least a portion of the simulated dataset (312); providing the training dataset (314) to a plurality of generator models (206) that each creates a separate graph (208) representing relationships in the training dataset (314), wherein at least one of the plurality of generator models (206) is a trainable generator model that is end-to-end differentiable and was previously trained with a generator model loss function; providing the graphs (208) created by the plurality of generator models to an ensemble model (210) that is a machine learning model (102) which creates a consensus graph (216) from the graphs (208) created by the plurality of generator models (206); and jointly training the trainable generator model (206A) and the ensemble model (210) on the training dataset (314) using an ensemble model loss function that includes the generator model loss function as a regularization term to minimize the difference between the consensus graph and the simulated graph.

Clause 18. The method of clause 17, wherein the training dataset (314) comprises at least one experimentally identified relationship.

Clause 19. The method of clause 17 or 18, wherein the biological data simulator (310) generates the simulated dataset using kinetic equations.

Clause 20. The method of clause 19, wherein the biological data simulator is one of BEELINE, SYNTREN, or SERGIO.

Clause 21. The method of any of clauses 17-20, wherein jointly training the trainable generator model and the ensemble model uses stochastic gradient descent.

Clause 22. Computer-readable storage media comprising instructions that when executed by a processor cause a computing device to perform the method of any of clauses 17-21.

### Conclusion

While certain example embodiments have been described, including the best mode known to the inventors for carrying out the invention, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. Skilled artisans will know how to employ such variations as appropriate, and the embodiments disclosed herein may be practiced otherwise than specifically described. The accompanying claims and their equivalents are intended to cover such forms or modifications as would fall within the scope and spirit of certain of the inventions disclosed herein.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different sensors).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Furthermore, references have been made to publications, patents and/or patent applications throughout this specification. Each of the cited references is individually incorporated herein by reference for its particular cited teachings as well as for all that it discloses.

1. A method for visualizing complex data relationships comprising:
receiving expression data;
providing the expression data to a first generator model that generates a first graph representing relationships in the expression data, wherein the first generator model is a trainable generator model that is end-to-end differentiable and was previously trained with a generator model loss function;
providing the expression data to a second generator model that, in parallel with the first generator model, generates a second graph representing relationships in the expression data;
providing the first graph and the second graph to an ensemble model that is a machine learning model and creates consensus relationship data from the first graph and the second graph, wherein the ensemble model and the first generator model are jointly trained with an ensemble model loss function that includes the generator model loss function as a regularization term; and
generating a consensus graph having nodes and edges from the consensus relationship data.

2. The method of claim 1, wherein the expression data is microarray data generated by single cell RNA sequencing or bulk sequencing.

3. The method of claim 2, further comprising generating expression data with a microarray.

4. The method of claim 1, wherein the first generator model is one of GLAD, uGLAD, Neural Graph Revealers (NGR), or GRNUlar.

5. The method of claim 1, wherein the second generator model is a fixed model that is not trainable.

6. The method of claim 5, wherein the second generator model is one of GENIE3 or GRNBoost2.

7. The method of claim 1, wherein the ensemble model learns a function for each edge in the consensus graph over the edges present in the first graph and the second graph.

8. The method of claim 7, wherein the ensemble model is an edge-selector neural network.

9. The method of claim 1, wherein, in the consensus graph, the nodes represent genes and the edges represents a regulatory relationship between a first one of the genes and a second one of the genes, wherein the first one of the genes is a transcription factor gene.

10. A system for visualizing complex data relationships comprising:
a processor;
memory coupled to the processor;
a data input component configured to receive expression data;
a plurality of generator models each configured to generate a graph representing relationships in the expression data, wherein at least one of the plurality of generator models is a trainable generator model that is end-to-end differentiable and was previously trained with a generator model loss function;
an ensemble model that is configured to create consensus relationship data from the graphs generated by the plurality of generator models, wherein the ensemble model is a machine learning model trained with an ensemble model loss function that includes the generator model loss function as a regularization term; and
a graph visualization component configured to generate a consensus graph having nodes and edges from the consensus relationship data.

11. The system of claim 10, further comprising a data simulator configured to generate a simulated dataset that includes a simulated graph and simulated expression data for use as a training dataset.

12. The system of claim 11, further comprising a training module configured to jointly train the trainable generator model and the ensemble model on the training dataset by minimizing the loss of the ensemble model loss function that includes the generator model loss function as a regularization term.

13. The system of claim 10, wherein the plurality of generator models includes a first trainable generator model that was previously trained with a first generator model loss function and a second trainable generator model that was previously trained with a second generator model loss function and the ensemble model is trained with an ensemble model loss function that includes the first generator model loss function as a first regularization term and the second generator model loss function as a second regularization term.

14. The system of claim 10, wherein the plurality of generator models are graph recovery models.

15. The system of claim 10, wherein the ensemble model learns a function for each edge in the consensus graph over the edges present in the graphs generated by the plurality of generator models.

16. A method for training a machine learning model to generate a visual representation of complex data comprising:
generating a simulated dataset with a data simulator, wherein the simulated dataset includes simulated expression data and a simulated graph representing relationships in the simulated expression data;
creating a training dataset that comprises at least a portion of the simulated dataset;
providing the training dataset to a plurality of generator models that each creates a separate graph representing relationships in the training dataset, wherein at least one of the plurality of generator models is a trainable generator model that is end-to-end differentiable and was previously trained with a generator model loss function;
providing the graphs created by the plurality of generator models to an ensemble model that is a machine learning model which creates a consensus graph from the graphs created by the plurality of generator models; and
jointly training the trainable generator model and the ensemble model on the training dataset using an ensemble model loss function that includes the generator model loss function as a regularization term to minimize the difference between the consensus graph and the simulated graph.

17. The method of claim 16, wherein the training dataset comprises at least one experimentally identified relationship.

18. The method of claim 16, wherein the data simulator generates the simulated dataset using kinetic equations.

19. The method of claim 16, wherein the data simulator is one of BEELINE, SYNTREN, or SERGIO.

20. The method of claim 16, wherein jointly training the trainable generator model and the ensemble model uses stochastic gradient descent.
